(19) United States
(12) Patent Application Publication
(10) Pub. No.: US 2021/0259278 A1
(43) Pub. Date: Aug. 26, 2021

(54) CHEWING GUM CONTAINING VITAMIN C

(71) Applicant: Reinhart JARISCH, Wien (AT)

(72) Inventor: Reinhart JARISCH, Wien (AT)

(21) Appl. No.: 17/254,127

(22) PCT Filed: Jun. 17, 2019
(86) PCT No.: PCT/AT2019/000017

§ 371 Date: Dec. 18, 2020

(30) Foreign Application Priority Data
Jun. 21, 2018 (AT) GM 131/2018

(51) Int. Cl. A23G 4/12; A61K 31/375; A61K 9/68

(52) U.S. Cl. CPC A23G 4/126; A61K 9/0058; A61K 31/375

(57) ABSTRACT

Chewing gum containing vitamin C for use in the treatment of nausea in humans.

The present invention relates to a chewing gum containing vitamin C for use in the treatment of nausea in humans.

Nausea in humans, for example when traveling by sea, on bus trips or car journeys in the back seat and when driving autonomously, on airplanes and in space travel, is sometimes due to a lack of vitamin C in the blood. The deficiency is associated with increased histamine levels, which in turn is associated with states of nausea. The administration of vitamin C has therefore been investigated and found to be effective for the treatment of nausea, for example in connection with seasickness (Impact of oral vitamin C on histamine levels and seasickness.; J Vestib Res. 2014; 24(4):281-8).

At the same time, these studies have shown that the effect of administering vitamin C alone could not reliably prevent the occurrence of nausea in all test subjects. The present invention is therefore based on the object of improving the antiemetic effect of vitamin C by providing a new and inventive dosage form.

The object of the invention is achieved by providing a chewing gum containing vitamin C for use in the treatment of nausea in humans. Chewing gum has a strong anti-emetic effect by stimulating the flow of saliva (Chewing gum for the treatment of nausea and vomiting: a pilot randomized controlled trial. Br. J. Anaesth. January, 118(1):83-89) and intensifies this effect of vitamin C, so that a highly effective preparation that is easy and enjoyable to consume can be made available. In addition, when using the vitamin C chewing gum according to the invention, unlike when it is taken in the form of effervescent tablets or dragees, the active ingredient is absorbed sublingually to a substantial extent, which leads to particularly rapid and complete absorption of the active ingredient. The dosage form as chewing gum is extremely durable, can easily be taken with you on trips and consumed discreetly.

Preferably, the chewing gum according to the present invention is for use in treating the nausea conditions associated with early pregnancy. A high histamine level has also been linked to an increased risk of miscarriage, so that chewing gum containing vitamin C also improves the circumstances of early pregnancy in this regard.

In an alternate preferred embodiment, the chewing gum of the present invention is for use in treating seasickness associated nausea.

A piece of chewing gum preferably contains vitamin C in amounts from 50 mg to 300 mg, preferably in amounts from 100 mg to 250 mg, particularly preferably in amounts from 150 mg to 200 mg. Such concentrations can be regarded as very high, but no side effects occur.

In order to optimize the suitability of the chewing gum according to the present invention with regard to a long shelf life and transportability, it is preferably provided that the chewing gum is coated.

In connection with the present invention, the term vitamin C includes not only L-(+)-ascorbic acid but also its derivatives with the same effect and thus substances that can be converted to L-(+)-ascorbic acid in the body, such as dehydroascorbic acid.

1-5. (canceled)

6. A Chewing gum containing vitamin C for use in the treatment of nausea in humans, characterized in that a piece of chewing gum contains vitamin C in amounts of 50 mg to 300 mg.

7. The chewing gum according to claim 6, for use in the treatment of nausea associated with early pregnancy.

8. The chewing gum according to claim 6, for use in the treatment of nausea associated with seasickness.

9. The chewing gum according to claim 6, characterized in that the chewing gum is coated.

10. The chewing gum according to claim 7, characterized in that the chewing gum is coated.

11. The chewing gum according to claim 8, characterized in that the chewing gum is coated.

12. The chewing gun according to claim 6, wherein the piece of chewing gum contains vitamin C in amounts of 100 mg to 250 mg.

13. The chewing gun according to claim 12, wherein the piece of chewing gum contains vitamin C in amounts of 150 mg to 200 mg.
